(19) United States
(12) Patent Application Publication
(10) Pub. No.: US 2024/0065631 A1
(43) Pub. Date: Feb. 29, 2024

(54) PRESSURE ADJUSTMENT FOR BIOMETRIC MEASUREMENT

(71) Applicant: QUALCOMM Incorporated, San Diego, CA (US)

(72) Inventors: Emily Kathryn BROOKS, Amherst, NY (US); John Keith SCHNEIDER, Williamsville, NY (US); Krishna BUSKA, San Diego, CA (US)

(21) Appl. No.: 17/822,307

(22) Filed: Aug. 25, 2022

(51) Int. Cl. A61B 5/00; G04G 21/02; G04G 9/00

(52) U.S. Cl. CPC A61B 5/6843; G04G 21/025; G04G 9/007; A61B 5/681; A61B 5/0095

(57) ABSTRACT

In some aspects, a user device may initiate a biometric measurement operation relating to a user of the user device. The user device may monitor, using a pressure sensor of the user device, whether a pressure at an interface between a body of the user and the user device is within a range used for obtaining data for a biometric measurement. The user device may provide an indication of whether the user is to adjust the pressure based on whether the pressure is within the range. The user device may obtain the data for the biometric measurement. Numerous other aspects are described.

### FIELD OF THE DISCLOSURE

Aspects of the present disclosure generally relate to biometric measurement and, for example, to pressure adjustment for biometric measurement.

### BACKGROUND

A wearable device may include a device that is incorporated into an item that can be worn in and/or on the human body, such as an article of clothing, an accessory, or the like. In some cases, the wearable device may be capable of performing a task typically performed by another type of device (e.g., a smart phone, a mobile phone, or a laptop computer) and/or a task that the other type of device does not typically perform (e.g., providing biofeedback, tracking a physiological function, or the like).

### SUMMARY

In some aspects, a user device includes a pressure sensor configured to detect a pressure at an interface between a body of a user and the user device. The user device may include an output component. The user device may include one or more processors configured to: monitor, in connection with a biometric measurement operation and using the pressure sensor, whether a pressure at the interface between the body of the user and the user device is within a range used for obtaining data for a biometric measurement; provide, via the output component, an indication of whether the user is to adjust the pressure based on whether the pressure is within the range; and obtain the data for the biometric measurement.

In some aspects, a method includes initiating, by a user device, a biometric measurement operation relating to a user of the user device. The method may include monitoring, by the user device in connection with the biometric measurement operation and using a pressure sensor of the user device, whether a pressure at an interface between a body of the user and the user device is within a range used for obtaining data for a biometric measurement. The method may include providing, by the user device via an output component of the user device, an indication of whether the user is to adjust the pressure based on whether the pressure is within the range. The method may include obtaining, by the user device, the data for the biometric measurement.

In some aspects, a user device includes means for initiating a biometric measurement operation relating to a user of the user device. The user device may include means for monitoring whether a pressure at an interface between a body of the user and the user device is within a range used for obtaining data for a biometric measurement. The user device may include means for providing an indication of whether the user is to adjust the pressure based on whether the pressure is within the range. The user device may include means for obtaining the data for the biometric measurement.

In some aspects, a non-transitory computer-readable medium storing a set of instructions includes one or more instructions that, when executed by one or more processors of a user device, cause the user device to: initiate a biometric measurement operation relating to a user of the user device. The one or more instructions, when executed by the one or more processors, may cause the user device to monitor, in connection with the biometric measurement operation and using a pressure sensor of the user device, whether a pressure at an interface between a body of the user and the user device is within a range used for obtaining data for a biometric measurement. The one or more instructions, when executed by the one or more processors, may cause the user device to provide, via an output component of the user device, an indication that the user is to increase or decrease the pressure to within the range. The one or more instructions, when executed by the one or more processors, may cause the user device to detect that the pressure is within the range used for obtaining the data for the biometric measurement. The one or more instructions, when executed by the one or more processors, may cause the user device to obtain the data for the biometric measurement based on detecting that the pressure is within the range.

Aspects generally include a method, apparatus, system, computer program product, non-transitory computer-readable medium, user device, user equipment, wireless communication device, and/or processing system as substantially described with reference to and as illustrated by the drawings and specification.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the scope of the appended claims. Characteristics of the concepts disclosed herein, both their organization and method of operation, together with associated advantages will be better understood from the following description when considered in connection with the accompanying figures. Each of the figures is provided for the purposes of illustration and description, and not as a definition of the limits of the claims.

### BRIEF DESCRIPTION OF THE DRAWINGS

So that the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects. The same reference numbers in different drawings may identify the same or similar elements.

FIG. 1 is a diagram of an example environment in which systems and/or methods described herein may be implemented.

FIG. 2 is a diagram illustrating example components of a device, in accordance with the present disclosure.

FIGS. 3A-3B are a front view and a back view, respectively, of an example user device.

FIGS. 4A-4C are diagrams of an example associated with pressure adjustment for biometric measurement.

FIG. 5 is a flowchart of an example process associated with pressure adjustment for biometric measurement.

FIG. 6 is a flowchart of an example process associated with pressure adjustment for biometric measurement.

### DETAILED DESCRIPTION

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. One skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

A wearable device may be used for obtaining a biometric measurement relating to a wearer (e.g., a user) of the wearable device. In one example, a smartwatch may be used for obtaining a blood pressure measurement of the wearer. Sometimes, an accuracy of the biometric measurement may be affected by a manner in which the wearable device is worn by the wearer. For example, a smartwatch may be worn at various positions along an arm of the wearer and/or a strap for the smartwatch may be tightened to the arm of the wearer with varying tightness. These variables affect a pressure between the smartwatch and the arm of the wearer, thereby affecting an accuracy of a biometric measurement, such as a blood pressure measurement, taken by the smartwatch. In particular, the smartwatch may utilize photoacoustic signal generation for blood pressure measurements or other biometric measurements, and variations in applied pressure may alter a received photoacoustic signal at a sensor of the smartwatch. As a result, the smartwatch may be unable to consistently take accurate and reliable blood pressure measurements or other biometric measurements. In some cases, this lack of consistency may result in measurements being taken repeatedly, thereby consuming excessive resources of the smartwatch (e.g., processing resources, memory resources, and/or battery resources, among other examples).

In some techniques and apparatuses described herein, a user device may monitor a pressure at an interface between the user device and a body (e.g., an arm) of a user of the user device, and the user device may provide an indication to the user of whether the pressure is to be adjusted to within a range used to obtain data for a biometric measurement. For example, the indication may be provided in a user interface presented on a display of the user device. In response to the indication, the user may increase, decrease, or maintain the pressure (e.g., by applying pressure to the user device or decreasing applied pressure to the user device). In this way, the user device may obtain an accurate biometric measurement. Moreover, the user device may obtain the accurate biometric measurement on a first attempt, thereby reducing the number of measurements that are taken and conserving resources of the user device (e.g., processing resources, memory resources, and/or battery resources, among other examples). Furthermore, the user device may obtain the accurate biometric measurement without the use of a pressure-adjusting air bladder in the user device, thereby improving a form factor of the user device and facilitating miniaturization.

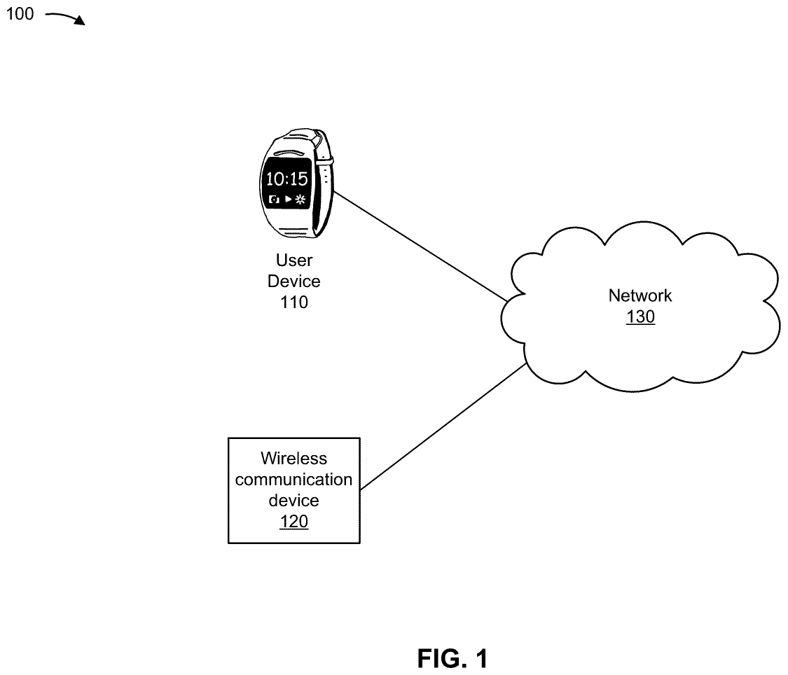

FIG. 1 is a diagram of an example environment 100 in which systems and/or methods described herein may be implemented. As shown in FIG. 1, environment 100 may include a user device 110, a wireless communication device 120, and a network 130. Devices of environment 100 may interconnect via wired connections, wireless connections, or a combination of wired and wireless connections.

The user device 110 may include one or more devices capable of receiving, generating, storing, processing, and/or providing information associated with pressure adjustment for biometric measurement, as described elsewhere herein. The user device 110 may include a communication device and/or a computing device. For example, the user device 110 may include a wireless communication device, a mobile phone, a wearable communication device (e.g., a smart wristwatch, a pair of smart eyeglasses, a head mounted display, or a virtual reality headset), or a similar type of device.

The wireless communication device 120 includes one or more devices capable of receiving, generating, storing, processing, providing, and/or routing information associated with biometric measurement, as described elsewhere herein. The wireless communication device 120 may include a communication device and/or a computing device. For example, the wireless communication device 120 may include a server, such as an application server, a client server, a web server, a database server, a host server, a proxy server, a virtual server (e.g., executing on computing hardware), or a server in a cloud computing system. In some aspects, the wireless communication device 120 includes computing hardware used in a cloud computing environment.

The network 130 may include one or more wired and/or wireless networks. For example, the network 130 may include a wireless wide area network (e.g., a cellular network or a public land mobile network), a local area network (e.g., a wired local area network or a wireless local area network (WLAN), such as a Wi-Fi network), a personal area network (e.g., a Bluetooth network), a near-field communication network, a telephone network, a private network, the Internet, and/or a combination of these or other types of networks. The network 130 enables communication among the devices of environment 100.

The number and arrangement of devices and networks shown in FIG. 1 are provided as an example. In practice, there may be additional devices and/or networks, fewer devices and/or networks, different devices and/or networks, or differently arranged devices and/or networks than those shown in FIG. 1. Furthermore, two or more devices shown in FIG. 1 may be implemented within a single device, or a single device shown in FIG. 1 may be implemented as multiple, distributed devices. Additionally, or alternatively, a set of devices (e.g., one or more devices) of environment 100 may perform one or more functions described as being performed by another set of devices of environment 100.

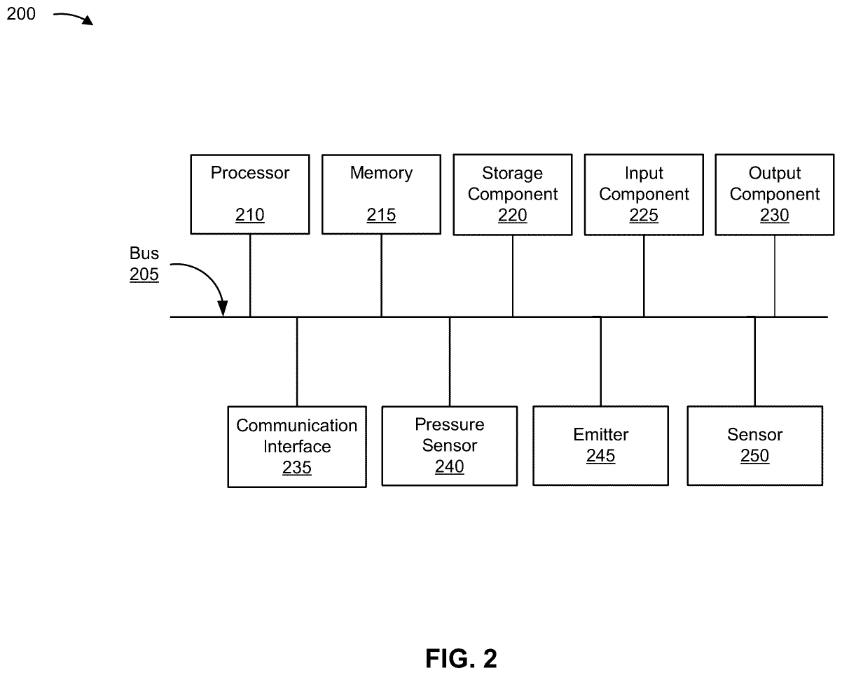

FIG. 2 is a diagram illustrating example components of a device 200, in accordance with the present disclosure. Device 200 may correspond to user device 110 and/or wireless communication device 120. In some aspects, user device 110 and/or wireless communication device 120 may include one or more devices 200 and/or one or more components of device 200. As shown in FIG. 2, device 200 may include a bus 205, a processor 210, a memory 215, a storage component 220, an input component 225, an output component 230, a communication interface 235, a pressure sensor 240, an emitter 245, and/or a sensor 250.

Bus 205 includes a component that permits communication among the components of device 200. Processor 210 is implemented in hardware, firmware, or a combination of hardware and software. Processor 210 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some aspects, processor 210 includes one or more processors capable of being programmed to perform a function. Memory 215 includes a random access memory (RAM), a read only memory (ROM), and/or another type of dynamic or static storage device (e.g., a flash memory, a magnetic memory, and/or an optical memory) that stores information and/or instructions for use by processor 210.

Storage component 220 stores information and/or software related to the operation and use of device 200. For example, storage component 220 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

Input component 225 includes a component that permits device 200 to receive information, such as via user input (e.g., a touch screen display, a keyboard, a keypad, a mouse, a button, a switch, and/or a microphone). Additionally, or alternatively, input component 225 may include a component for determining a position or a location of device 200 (e.g., a global positioning system (GPS) component or a global navigation satellite system (GNSS) component) and/or a sensor for sensing information (e.g., an accelerometer, a gyroscope, or another type of position or environment sensor). Output component 230 includes a component that provides output information from device 200 (e.g., a display, a speaker, a haptic feedback component, and/or an audio or visual indicator).

Communication interface 235 includes a transceiver-like component (e.g., a transceiver and/or a separate receiver and transmitter) that enables device 200 to communicate with other devices, such as via a wired connection, a wireless connection, or a combination of wired and wireless connections. Communication interface 235 may permit device 200 to receive information from another device and/or provide information to another device. For example, communication interface 235 may include an Ethernet interface, an optical interface, a coaxial interface, an infrared interface, a radio frequency interface, a universal serial bus (USB) interface, a wireless local area interface (e.g., a Wi-Fi interface), and/or a cellular network interface.

Pressure sensor 240 includes one or more devices capable of detecting a pressure at an interface between device 200 and a body of a user of device 200. Pressure sensor 240 may include a piezoresistive pressure sensor, a piezoelectric pressure sensor, a capacitive pressure sensor, an electromagnetic pressure sensor, or another type of pressure sensor.

Emitter 245 includes a component configured to emit a signal from device 200. For example, emitter 245 may include an optical emitter configured to emit an optical signal. The optical emitter may include one or more light emitting diodes (LEDs), one or more vertical cavity surface emitting lasers (VCSELs), one or more edge-emitting lasers, or another type of light-emitting device. The optical signal may use visible light, near-infrared light, infrared light, and/or ultraviolet light. In some aspects, emitter 245 may include a sound emitter configured to emit a sound wave (e.g., using audible sound, infrasound, and/or ultrasound). In some aspects, emitter 245 may include a radio transmitter configured to transmit a radio frequency (RF) signal.

Sensor 250 includes one or more devices capable of detecting a signal received at device 200. For example, sensor 250 may include a sound sensor (e.g., an audible sound sensor, an infrasound sensor, and/or an ultrasound sensor). In particular, sensor 250 may be configured to detect a sound wave that is generated by the photoacoustic effect from an optical signal emitted by emitter 245. In some aspects, sensor 250 may include a photosensor. In some aspects, sensor 250 may include a radio receiver.

Device 200 may perform one or more processes described herein. Device 200 may perform these processes based on processor 210 executing software instructions stored by a non-transitory computer-readable medium, such as memory 215 and/or storage component 220. A computer-readable medium is defined herein as a non-transitory memory device. A memory device includes memory space within a single physical storage device or memory space spread across multiple physical storage devices.

Software instructions may be read into memory 215 and/or storage component 220 from another computer-readable medium or from another device via communication interface 235. When executed, software instructions stored in memory 215 and/or storage component 220 may cause processor 210 to perform one or more processes described herein. Additionally, or alternatively, hardwired circuitry may be used in place of or in combination with software instructions to perform one or more processes described herein. Thus, aspects described herein are not limited to any specific combination of hardware circuitry and software.

In some aspects, device 200 includes means for performing one or more processes described herein and/or means for performing one or more operations of the processes described herein. For example, device 200 may include means for initiating a biometric measurement operation relating to a user of a user device; means for monitoring whether a pressure at an interface between a body of the user and the user device is within a range used for obtaining data for a biometric measurement; means for providing an indication of whether the user is to adjust the pressure based on whether the pressure is within the range; means for obtaining the data for the biometric measurement; or the like. In some aspects, such means may include one or more components of device 200 described in connection with FIG. 2, such as bus 205, processor 210, memory 215, storage component 220, input component 225, output component 230, communication interface 235, pressure sensor 240, emitter 245, and/or sensor 250.

The number and arrangement of components shown in FIG. 2 are provided as an example. In practice, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

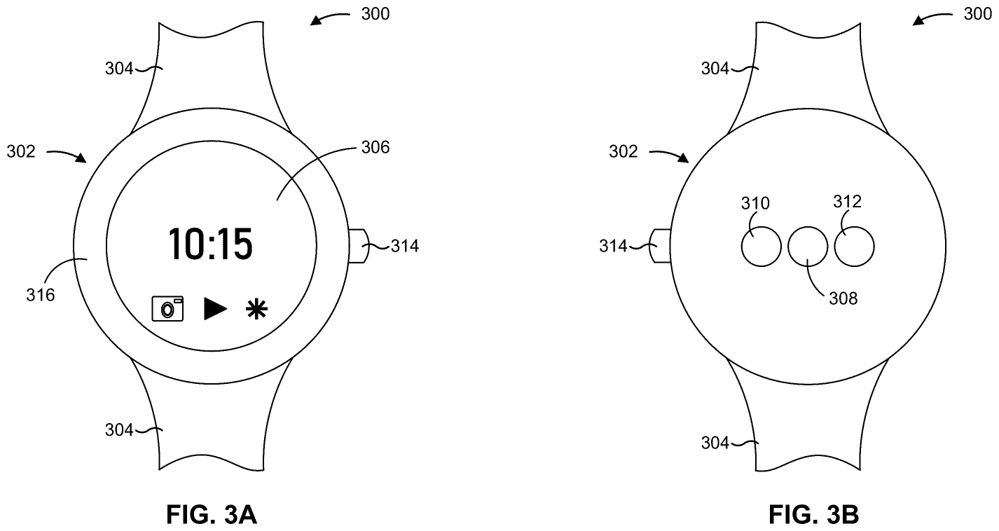

FIGS. 3A-3B are a front view and a back view, respectively, of an example user device 300 (e.g., which may correspond to user device 110). As described herein, the user device 300 may be a wearable device, such as a wearable device configured for attachment to an arm of a user. For example, in FIGS. 3A-3B, the user device 300 is shown as a smart wristwatch. However, in some aspects, the user device 300 may be another type of wearable device (e.g., a smart arm cuff or a smart ring), or the user device 300 may be a device that is not configured for wearing.

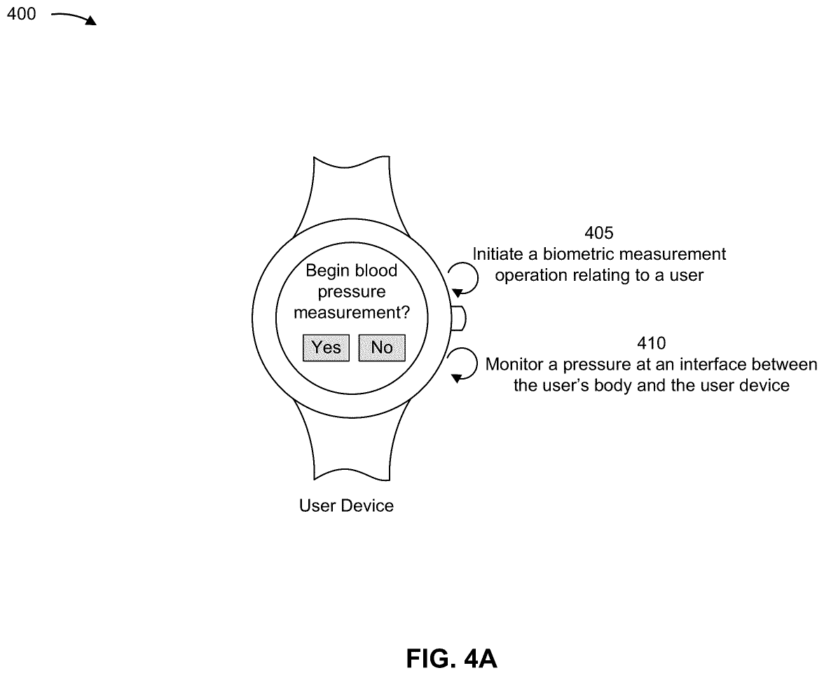

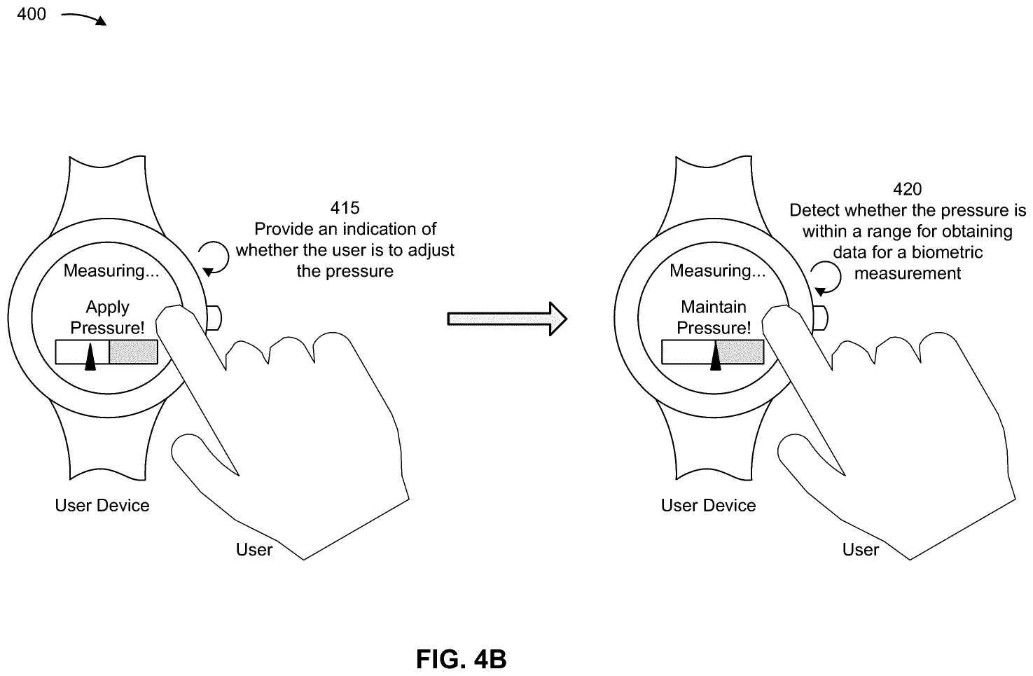

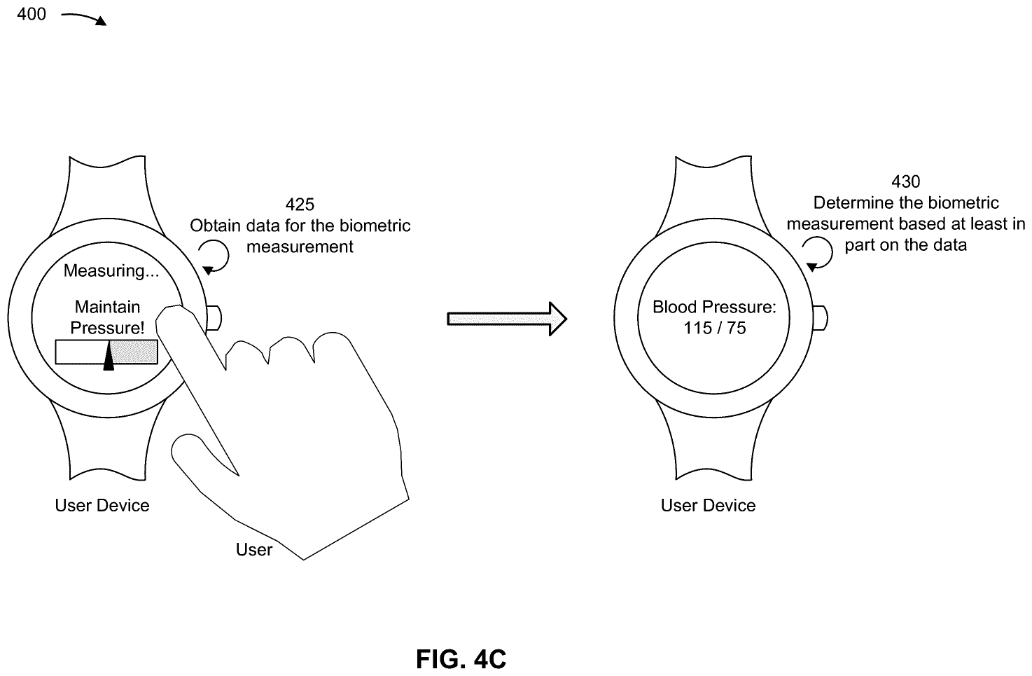

As shown, the user device 300 may include a device component 302 and at least one strap 304 (shown as two straps 304 that may be joined together by a buckle, a clasp, or the like) connected to the device component 302. The strap(s) 304 (e.g., a wristband) may enable the user device 300 for attachment to an arm (e.g., a wrist or a finger) of a user. The user device 300 (e.g., the device component 302) may include one or more processors configured to perform operation described herein, such as operations described in connection with FIGS. 4A-4C.

As shown in FIG. 3A, a first side of the user device 300 (e.g., a first side of the device component 302) may include a display 306 (e.g., corresponding to output component 230). The display 306 may be configured to present a user interface. For example, the user interface may include an input element that enables initiation of a biometric measurement operation (e.g., a blood pressure measurement operation).

As shown in FIG. 3B, a second side of the user device 300 (e.g., a second side of the device component 302), opposite the first side, may include a pressure sensor 308 (e.g., corresponding to pressure sensor 240). The pressure sensor 308 may be configured to detect a pressure at an interface between a body (e.g., a wrist) of a user (e.g., a wearer) of the user device 300 and the user device 300. The interface may be a point of contact between the second side of the user device 300 and the body of the user. For example, the pressure sensor 308, or a portion thereof, may project from a surface of the device component 302, or the pressure sensor 308 may be flush with the surface of the device component 302. In this way, when the user device 300 is worn by the user, the pressure sensor 308 contacts the body of the user. In some aspects, the second side of the user device 300 may further include an emitter 310 (e.g., corresponding to emitter 245) to emit a signal at the body of the user and/or a sensor 312 (e.g., corresponding to sensor 250) to detect a reflection of an emitted signal or a signal generated by an emitted signal. For example, the emitter 310 may be configured to emit an optical signal (e.g., a near-infrared or infrared optical signal) at the body of the user, and the sensor 312 may be configured to detect a sound wave (e.g., an ultrasound wave) that is generated by the photoacoustic effect when the optical signal interacts with the body of the user. As another example, the emitter 310 may be configured to emit an optical signal (e.g., a near-infrared or infrared optical signal) at the body of the user, and the sensor 312 may be configured to detect light reflected back from the body (e.g., blood) of the user.

The user device 300 may include a rotatable element that is configured to cause actuation (e.g., projection or retraction) of the pressure sensor 308 (as well as the emitter 310 and/or the sensor 312) to thereby adjust the pressure at the interface between the body of the user and the user device 300 (e.g., by actuating the pressure sensor 308 toward or away from the user's body). Actuation of the pressure sensor 308 may include actuation of a surface (e.g., a plate or a substrate) that supports the pressure sensor 308 (as well as the emitter 310 and/or the sensor 312). In some aspects, the rotatable element may be mechanically linked to the pressure sensor 308, or the surface that includes the pressure sensor 308, by one or more gears, one or more screws, or the like, such that rotation of the rotatable element causes actuation of the pressure sensor 308 via the mechanical linkage. In some aspects, the rotatable element may be a control for actuation of the pressure sensor 308, and actuation of the pressure sensor 308 may be achieved via an actuation component (e.g., a linear actuator) of the device component 302. In some aspects, the rotatable element may be a knob 314 (also referred to as a “crown” in the case of a smart watch) that is connected to the device component 302 of the user device 300. Additionally, or alternatively, the rotatable element may be a bezel 316 that surrounds the display 306.

As indicated above, FIGS. 3A-3B are provided as an example. Other examples may differ from what is described with regard to FIGS. 3A-3B.

FIGS. 4A-4C are diagrams of an example 400 associated with pressure adjustment for biometric measurement. As shown in FIGS. 4A-4C, example 400 includes a user device, which is described in more detail in connection with FIGS. 1-2 and 3A-3B. For example, the user device may correspond to user device 300.

As shown in FIG. 4A, and by reference number 405, the user device may initiate a biometric measurement operation relating to a user of the user device. The user of the user device may be wearing the user device (e.g., on an arm of the user). The biometric measurement operation may be a blood pressure measurement operation. Additionally, or alternatively, the biometric measurement operation may be a heart rate measurement operation, a heart rate variability measurement operation, and/or a pulse oxygen measurement operation, among other examples. The user device may initiate the biometric measurement operation periodically, responsive to a request from the user (e.g., responsive to an input to the user device indicating a request to initiate the biometric measurement operation), or responsive to a triggering event.

As shown by reference number 410, in connection with the biometric measurement operation, the user device may monitor a pressure at an interface between a body (e.g., an arm) of the user and the user device. For example, the user device may monitor whether the pressure at the interface between the body of the user and the user device is within a range (e.g., about 30 millimetres of mercury (mmHg) to about 40 mmHg) used for obtaining data for a biometric measurement (e.g., a blood pressure measurement, a heart rate measurement, a heart rate variability measurement, and/or a pulse oxygen measurement, among other examples). The user device may monitor the pressure using a pressure sensor of the user device (e.g., pressure sensor 308).

As shown in FIG. 4B, and by reference number 415, the user device may provide an indication of whether the user is to adjust the pressure based on whether the pressure is within the range used for obtaining the data. In particular, the user device may provide, based on whether the pressure is within the range, an indication that the user is to increase the pressure to within the range, decrease the pressure to within the range, or maintain the pressure within the range. The user device may provide the indication in real time as the user device is monitoring the pressure.

The user device may provide the indication via an output component of the user device. For example, the output component may be a display (e.g., display 306). Here, the indication may be provided by a user interface element of a user interface presented on the display. The user interface element may include a gauge (e.g., to show a difference between a current pressure and the range) or color indicators (e.g., a red color indicator to indicate that a current pressure is outside of the range or a green color indicator to indicate that a current pressure is within the range). As another example, the output component may be a speaker. Here, the indication may be provided by one or more sounds (e.g., a first sound to indicate that a current pressure is to be increased, a second sound to indicate that a current pressure is to be decreased, or a third sound to indicate that a current pressure is to be maintained). As a further example, the output component may be a vibration component. Here, the indication may be provided by one or more vibration patterns (e.g., a first pattern to indicate that a current pressure is to be increased, a second pattern to indicate that a current pressure is to be decreased, or a third pattern to indicate that a current pressure is to be maintained).

Based on the indication, the user may adjust the pressure using one or more techniques. In one technique, the user may apply pressure to the user device (e.g., to the display of the user device) using a hand or a finger, or the user may decrease applied pressure to the user device. In another technique, the user may adjust a tightness of a strap (e.g., strap 304) of the user device. In an additional technique, the user may pull on the strap or insert a finger between the strap and the user's body (e.g., the user's arm). In a further technique, the user may rotate a rotatable element of the user device (e.g., knob 314 and/or bezel 316) that actuates the pressure sensor toward or away from the user's body. In another technique, the user may adjust a longitudinal position of the user device on the user's arm (e.g., toward the user's hand to decrease the pressure or toward the user's elbow to increase the pressure), the user's leg, or the like. In some aspects, the indication may indicate a manner in which the user is to adjust the pressure (e.g., the indication may indicate that the user is to employ one or more of the aforementioned techniques for adjusting the pressure). Adjustment of the pressure in accordance with the indication ensures that the user device can obtain multiple measurements at the same pressure.

In some aspects, the user device may provide the indication independent of the biometric measurement operation. For example, the user device may provide the indication while the user device is being attached to the user's body. Here, for example, the indication may indicate that the strap is to be tightened to a particular tightness, that the longitudinal position of the user device on the user's body is to be adjusted, or the like, to achieve pressure within the range for obtaining the data for the biometric measurement. The user device may detect that the user device is being attached to the user's body based on detecting (e.g., using a gyroscope and/or accelerometer) movement of the user device indicative of attachment.

As shown by reference number 420, the user device may detect whether the pressure at the interface between the user's body and the user device is within the range for obtaining the data for the biometric measurement. For example, the user device may detect whether the pressure is within the range based on monitoring the pressure. As described above, the user performing one or more techniques to adjust the pressure based on the indication may adjust the pressure to within the range.

As shown in FIG. 4C, and by reference number 425, the user device may obtain the data for the biometric measurement. For example, the user device may obtain the data based on detecting that the pressure is within the range. In some aspects, the user device may obtain the data based on providing the indication. That is, the user device may obtain the data while providing the indication. In some aspects, the user device may obtain the data using photoacoustic signal generation. For example, the user device may emit an optical signal (e.g., using emitter 310) at the user's body that interacts with the user's body and generates a sound wave. Continuing with the example, the user device may detect the sound wave (e.g., using sensor 312), and the sound wave may provide the data for the biometric measurement. Additionally, or alternatively, to obtain the data, the user device may emit an optical signal at the user's body and detect a reflection of the optical signal (e.g., where the reflection provides the data), emit a sound signal at the user's body and detect a reflection of the sound signal (e.g., where the reflection provides the data), and/or emit an RF signal at the user's body and detect a reflection of the RF signal (e.g., where the reflection provides the data). In some aspects, the data may indicate a plethysmogram (e.g., a photoplethysmogram (PPG) or a photoacousticplethysmogram (PAPG)).

In some cases, the pressure at the interface between the user's body and the user device may alternate between being within the range and being outside of the range while the user device is obtaining the data. For example, the data for the biometric measurement that is obtained may include first data for the biometric measurement that is obtained while the pressure is outside of the range and second data for the biometric measurement that is obtained while the pressure is within the range. The user device may filter the data based on whether the data is obtained while the pressure is outside of the range or while the pressure is within the range (which the user device can ascertain based on monitoring the pressure). Accordingly, the user device may discard the first data and retain the second data for further processing (e.g., for determining the biometric measurement based on the second data regardless of the first data).

Additionally, or alternatively, where the pressure at the interface between the user's body and the user device alternates between being within the range and being outside of the range while the user device is obtaining the data, the user device may adjust an intensity of an emitted signal. That is, the user device may adjust the intensity of the emitted signal based on an amount by which the pressure is outside of the range (which the user device can ascertain based on monitoring the pressure). For example, to obtain the data for the biometric measurement, the user device may emit an optical signal at the user's body, and the user device may adjust an intensity of the optical signal based on an amount by which the pressure is outside of the range. As an example, if the pressure is lower than the range, the user device may increase the intensity of the optical signal, and if the pressure is higher than the range, the user device may decrease the intensity of the optical signal. In this way, the data that is obtained by the user device is consistent across pressure differences, and an acceptable range for obtaining the data can be widened. In some aspects, in a similar manner as described above, the user device may adjust the intensity of an optical signal used for optical-based data collection, of an acoustic signal used for sound-based data collection, and/or of an RF signal used for radio-based data collection.

As shown by reference number 430, the user device my determine the biometric measurement based at least in part on the data that is obtained. For example, the user device may determine the biometric measurement based on the data alone or based on the data in combination with other data. The user device may determine the biometric measurement by processing the data using one or more algorithms and/or one or more machine learning models. As described above, the biometric measurement may be a blood pressure measurement, a heart rate measurement, a heart rate variability measurement, and/or a pulse oxygen measurement, among other examples.

In some aspects, another device (e.g., wireless communication device 120) may determine the biometric measurement based at least in part on the data that is obtained by the user device. For example, the user device may transmit the data to the other device, and the other device may transmit information indicating the biometric measurement to the user device.

The user device provides an indication of the biometric measurement to the user. In some aspects, the user device may present in the user interface on the display of the user device information indicating the biometric measurement. Additionally, or alternatively, the user device may transmit a message (e.g., an email message) that includes information indicating the biometric measurement. Additionally, or alternatively, the user device may record the biometric measurement in a log or another data structure for subsequent retrieval by the user.

In this way, the user device may facilitate a relatively constant pressure at the interface between the user device and the user's body to thereby obtain a relatively stable signal. Using this stable signal improves the accuracy of the biometric measurement, thereby reducing the number of measurements that need to be taken and conserving resources of the user device.

As indicated above, FIGS. 4A-4C are provided as an example. Other examples may differ from what is described with regard to FIGS. 4A-4C.

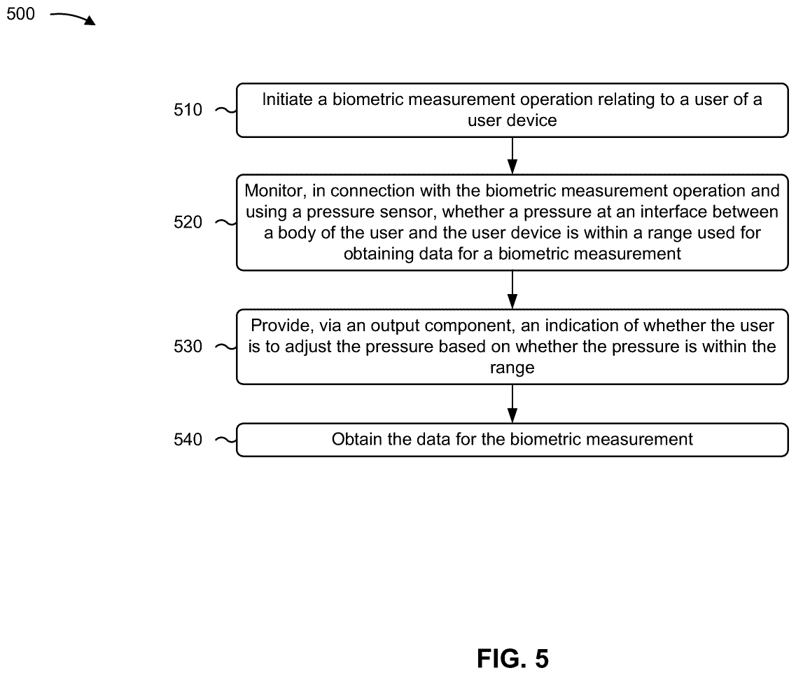

FIG. 5 is a flowchart of an example process 500 associated with pressure adjustment for biometric measurement. In some aspects, one or more process blocks of FIG. 5 are performed by a user device (e.g., user device 110). In some aspects, one or more process blocks of FIG. 5 are performed by another device or a group of devices separate from or including the user device, such as a wireless communication device (e.g., wireless communication device 120). Additionally, or alternatively, one or more process blocks of FIG. 5 may be performed by one or more components of device 200, such as processor 210, memory 215, storage component 220, input component 225, output component 230, communication interface 235, pressure sensor 240, emitter 245, and/or sensor 250.

As shown in FIG. 5, process 500 may include initiating a biometric measurement operation relating to a user of a user device (block 510). For example, the user device may initiate a biometric measurement operation relating to a user of the user device, as described above.

As further shown in FIG. 5, process 500 may include monitoring, using a pressure sensor, whether a pressure at an interface between a body of the user and the user device is within a range used for obtaining data for a biometric measurement (block 520). For example, the user device may monitor, using a pressure sensor of the user device, whether a pressure at an interface between a body of the user and the user device is within a range used for obtaining data for a biometric measurement, as described above.

As further shown in FIG. 5, process 500 may include providing an indication of whether the user is to adjust the pressure based on whether the pressure is within the range (block 530). For example, the user device may provide an indication of whether the user is to adjust the pressure based on whether the pressure is within the range, as described above.

As further shown in FIG. 5, process 500 may include obtaining the data for the biometric measurement (block 540). For example, the user device may obtain the data for the biometric measurement, as described above.

Process 500 may include additional aspects, such as any single aspect or any combination of aspects described below and/or in connection with one or more other processes described elsewhere herein.

In a first aspect, the data is obtained using photoacoustic signal generation.

In a second aspect, alone or in combination with the first aspect, the data that is obtained includes first data obtained while the pressure is outside of the range and second data obtained while the pressure is within the range, and process 500 further includes determining the biometric measurement based on the second data regardless of the first data.

In a third aspect, alone or in combination with one or more of the first and second aspects, obtaining the data includes emitting an optical signal at the body of the user, and adjusting an intensity of the optical signal based on an amount by which the pressure is outside of the range.

In a fourth aspect, alone or in combination with one or more of the first through third aspects, process 500 further includes determining the biometric measurement based at least in part on the data.

In a fifth aspect, alone or in combination with one or more of the first through fourth aspects, the output component is a display of the user device, a speaker of the user device, or a vibration component of the user device.

In a sixth aspect, alone or in combination with one or more of the first through fifth aspects, the user device is a wearable device configured to be worn on an arm of the user.

In a seventh aspect, alone or in combination with one or more of the first through sixth aspects, the biometric measurement is a blood pressure measurement, a heart rate measurement, a heart rate variability measurement, or a pulse oxygen measurement.

Although FIG. 5 shows example blocks of process 500, in some aspects, process 500 includes additional blocks, fewer blocks, different blocks, or differently arranged blocks than those depicted in FIG. 5. Additionally, or alternatively, two or more of the blocks of process 500 may be performed in parallel.

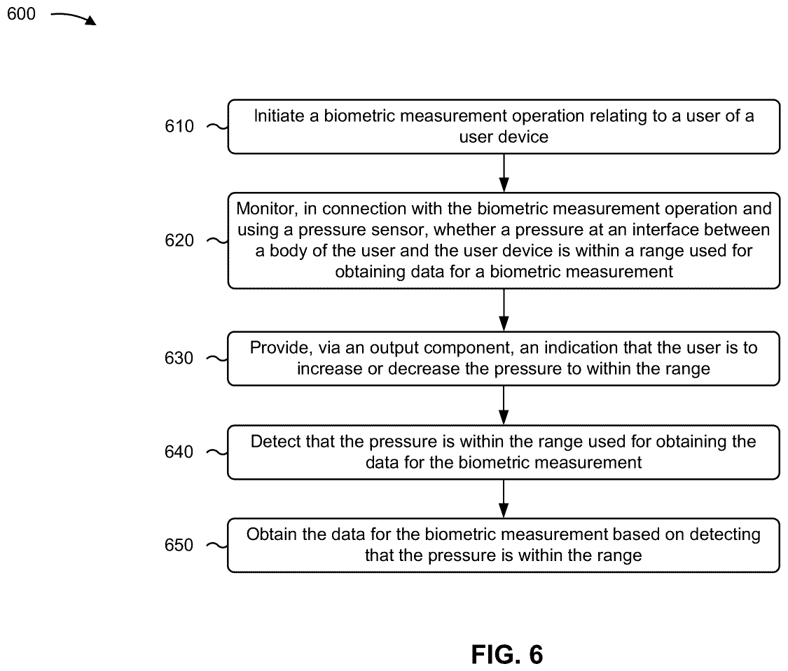

FIG. 6 is a flowchart of an example process 600 associated with pressure adjustment for biometric measurement. In some aspects, one or more process blocks of FIG. 6 are performed by a user device (e.g., user device 110). In some aspects, one or more process blocks of FIG. 6 are performed by another device or a group of devices separate from or including the user device, such as a wireless communication device (e.g., wireless communication device 120). Additionally, or alternatively, one or more process blocks of FIG. 6 may be performed by one or more components of device 200, such as processor 210, memory 215, storage component 220, input component 225, output component 230, communication interface 235, pressure sensor 240, emitter 245, and/or sensor 250.

As shown in FIG. 6, process 600 may include initiating a biometric measurement operation relating to a user of a user device (block 610). For example, the user device may initiate a biometric measurement operation relating to a user of the user device, as described above.

As further shown in FIG. 6, process 600 may include monitoring, in connection with the biometric measurement operation and using a pressure sensor, whether a pressure at an interface between a body of the user and the user device is within a range used for obtaining data for a biometric measurement (block 620). For example, the user device may monitor, in connection with the biometric measurement operation and using a pressure sensor of the user device, whether a pressure at an interface between a body of the user and the user device is within a range used for obtaining data for a biometric measurement, as described above.

As further shown in FIG. 6, process 600 may include providing, via an output component, an indication that the user is to increase or decrease the pressure to within the range (block 630). For example, the user device may provide, via an output component of the user device, an indication that the user is to increase or decrease the pressure to within the range, as described above.

As further shown in FIG. 6, process 600 may include detecting that the pressure is within the range used for obtaining the data for the biometric measurement (block 640). For example, the user device may detect that the pressure is within the range used for obtaining the data for the biometric measurement, as described above.

As further shown in FIG. 6, process 600 may include obtaining the data for the biometric measurement based on detecting that the pressure is within the range (block 650). For example, the user device may obtain the data for the biometric measurement based on detecting that the pressure is within the range, as described above.

Process 600 may include additional aspects, such as any single aspect or any combination of aspects described below and/or in connection with one or more other processes described elsewhere herein.

In a first aspect, obtaining the data for the biometric measurement includes obtaining the data for the biometric measurement using photoacoustic signal generation.

In a second aspect, alone or in combination with the first aspect, process 600 further includes determining the biometric measurement based at least in part on the data.

In a third aspect, alone or in combination with one or more of the first and second aspects, the output component is a display, a speaker, or a vibration component.

Although FIG. 6 shows example blocks of process 600, in some aspects, process 600 includes additional blocks, fewer blocks, different blocks, or differently arranged blocks than those depicted in FIG. 6. Additionally, or alternatively, two or more of the blocks of process 600 may be performed in parallel.

The following provides an overview of some Aspects of the present disclosure:

Aspect 1: A user device, comprising: a pressure sensor configured to detect a pressure at an interface between a body of a user and the user device; an output component; and one or more processors configured to: monitor, in connection with a biometric measurement operation and using the pressure sensor, whether a pressure at the interface between the body of the user and the user device is within a range used for obtaining data for a biometric measurement; provide, via the output component, an indication of whether the user is to adjust the pressure based on whether the pressure is within the range; and obtain the data for the biometric measurement.

Aspect 2: The user device of Aspect 1, wherein a first side of the user device includes a display, and wherein a second side of the user device, opposite the first side, includes the pressure sensor.

Aspect 3: The user device of any of Aspects 1-2, wherein the user device is a wearable device configured for attachment to an arm of the user, and wherein the user device further comprises a rotatable element configured to cause actuation of the pressure sensor to adjust the pressure at the interface between the body of the user and the user device.

Aspect 4: The user device of Aspect 3, wherein the rotatable element is a knob, or a bezel that surrounds a display of the user device.

Aspect 5: The user device of any of Aspects 1-4, wherein the one or more processors, to obtain the data for the biometric measurement, are configured to: obtain the data for the biometric measurement using photoacoustic signal generation.

Aspect 6: The user device of any of Aspects 1-5, wherein the one or more processors, to obtain the data for the biometric measurement, are configured to: obtain first data for the biometric measurement while the pressure is outside of the range and second data for the biometric measurement while the pressure is within the range; and determine the biometric measurement based on the second data regardless of the first data.

Aspect 7: The user device of any of Aspects 1-6, wherein the one or more processors, to obtain the data for the biometric measurement, are configured to: cause emission of an optical signal at the body of the user; and adjust an intensity of the optical signal based on an amount by which the pressure is outside of the range.

Aspect 8: The user device of any of Aspects 1-7, wherein the one or more processors are further configured to: determine the biometric measurement based at least in part on the data.

Aspect 9: The user device of any of Aspects 1-8, wherein the output component is a display, a speaker, or a vibration component.

Aspect 10: The user device of any of Aspects 1-9, wherein the user device further comprises: at least one strap to enable the user device to be worn on an arm of the user.

Aspect 11: The user device of any of Aspects 1-10, wherein the biometric measurement is a blood pressure measurement, a heart rate measurement, a heart rate variability measurement, or a pulse oxygen measurement.

Aspect 12: A method, comprising: initiating, by a user device, a biometric measurement operation relating to a user of the user device; monitoring, by the user device in connection with the biometric measurement operation and using a pressure sensor of the user device, whether a pressure at an interface between a body of the user and the user device is within a range used for obtaining data for a biometric measurement; providing, by the user device via an output component of the user device, an indication of whether the user is to adjust the pressure based on whether the pressure is within the range; and obtaining, by the user device, the data for the biometric measurement.

Aspect 13: The method of Aspect 12, wherein the data is obtained using photoacoustic signal generation.

Aspect 14: The method of any of Aspects 12-13, wherein the data that is obtained includes first data obtained while the pressure is outside of the range and second data obtained while the pressure is within the range, and wherein the method further comprises: determining the biometric measurement based on the second data regardless of the first data.

Aspect 15: The method of any of Aspects 12-14, wherein obtaining the data comprises: emitting an optical signal at the body of the user; and adjusting an intensity of the optical signal based on an amount by which the pressure is outside of the range.

Aspect 16: The method of any of Aspects 12-15, further comprising: determining the biometric measurement based at least in part on the data.

Aspect 17: The method of any of Aspects 12-16, wherein the output component is a display of the user device, a speaker of the user device, or a vibration component of the user device.

Aspect 18: The method of any of Aspects 12-17, wherein the user device is a wearable device configured to be worn on an arm of the user.

Aspect 19: The method of any of Aspects 12-18, wherein the biometric measurement is a blood pressure measurement, a heart rate measurement, a heart rate variability measurement, or a pulse oxygen measurement.

Aspect 20: A user device, comprising: means for initiating a biometric measurement operation relating to a user of the user device; means for monitoring whether a pressure at an interface between a body of the user and the user device is within a range used for obtaining data for a biometric measurement; means for providing an indication of whether the user is to adjust the pressure based on whether the pressure is within the range; and means for obtaining the data for the biometric measurement.

Aspect 21: The user device of Aspect 20, wherein the means for obtaining the data for the biometric measurement include: means for obtaining the data for the biometric measurement using photoacoustic signal generation.

Aspect 22: The user device of any of Aspects 20-21, wherein the means for obtaining the data for the biometric measurement include: means for obtaining first data for the biometric measurement while the pressure is outside of the range and second data for the biometric measurement while the pressure is within the range; and means for determining the biometric measurement based on the second data regardless of the first data.

Aspect 23: The user device of any of Aspects 20-22, wherein the means for obtaining the data for the biometric measurement include: means for emitting an optical signal at the body of the user; and means for adjusting an intensity of the optical signal based on an amount by which the pressure is outside of the range.

Aspect 24: The user device of any of Aspects 20-23, further comprising: means for determining the biometric measurement based at least in part on the data.

Aspect 25: The user device of any of Aspects 20-24, wherein the means for providing include a display of the user device, a speaker of the user device, or a vibration component of the user device.

Aspect 26: The user device of any of Aspects 20-25, wherein the means for monitoring include a pressure sensor of the user device.

Aspect 27: A non-transitory computer-readable medium storing a set of instructions, the set of instructions comprising: one or more instructions that, when executed by one or more processors of a user device, cause the user device to: initiate a biometric measurement operation relating to a user of the user device; monitor, in connection with the biometric measurement operation and using a pressure sensor of the user device, whether a pressure at an interface between a body of the user and the user device is within a range used for obtaining data for a biometric measurement; provide, via an output component of the user device, an indication that the user is to increase or decrease the pressure to within the range; detect that the pressure is within the range used for obtaining the data for the biometric measurement; and obtain the data for the biometric measurement based on detecting that the pressure is within the range.

Aspect 28: The non-transitory computer-readable medium of Aspect 27, wherein the one or more instructions, that cause the user device to obtain the data for the biometric measurement, cause the user device to: obtain the data for the biometric measurement using photoacoustic signal generation.

Aspect 29: The non-transitory computer-readable medium of any of Aspects 27-28, wherein the one or more instructions further cause the user device to: determine the biometric measurement based at least in part on the data.

Aspect 30: The non-transitory computer-readable medium of any of Aspects 27-29, wherein the output component is a display, a speaker, or a vibration component.

Aspect 31: An apparatus, comprising a processor; memory coupled with the processor; and instructions stored in the memory and executable by the processor to cause the apparatus to perform the steps performed by the one or more processors of one or more of Aspects 1-11.

Aspect 32: A method comprising the steps performed by the one or more processors of one or more of Aspects 1-11.

Aspect 33: An apparatus, comprising at least one means for performing the steps performed by the one or more processors of one or more of Aspects 1-11.

Aspect 34: A non-transitory computer-readable medium storing code, the code comprising instructions executable by a processor to perform the steps performed by the one or more processors of one or more of Aspects 1-11.

Aspect 35: A non-transitory computer-readable medium storing a set of instructions, the set of instructions comprising one or more instructions that, when executed by one or more processors of a device, cause the device to perform the steps performed by the one or more processors of one or more of Aspects 1-11.

Aspect 36: An apparatus, comprising a processor; memory coupled with the processor; and instructions stored in the memory and executable by the processor to cause the apparatus to perform the method of one or more of Aspects 12-19.

Aspect 37: A device, comprising a memory and one or more processors coupled to the memory, the one or more processors configured to perform the method of one or more of Aspects 12-19.

Aspect 38: An apparatus, comprising at least one means for performing the method of one or more of Aspects 12-19.

Aspect 39: A non-transitory computer-readable medium storing code, the code comprising instructions executable by a processor to perform the method of one or more of Aspects 12-19.

Aspect 40: A non-transitory computer-readable medium storing a set of instructions, the set of instructions comprising one or more instructions that, when executed by one or more processors of a device, cause the device to perform the method of one or more of Aspects 12-19.

Aspect 41: An apparatus, comprising a processor; memory coupled with the processor; and instructions stored in the memory and executable by the processor to cause the apparatus to perform the steps performed by the one or more processors of one or more of Aspects 20-26.

Aspect 42: A method comprising the steps performed by the one or more processors of one or more of Aspects 20-26.

Aspect 43: An apparatus, comprising at least one means for performing the steps performed by the one or more processors of one or more of Aspects 20-26.

Aspect 44: A non-transitory computer-readable medium storing code, the code comprising instructions executable by a processor to perform the steps performed by the one or more processors of one or more of Aspects 20-26.

Aspect 45: A non-transitory computer-readable medium storing a set of instructions, the set of instructions comprising one or more instructions that, when executed by one or more processors of a device, cause the device to perform the steps performed by the one or more processors of one or more of Aspects 20-26.

Aspect 46: An apparatus, comprising a processor; memory coupled with the processor; and instructions stored in the memory and executable by the processor to cause the apparatus to perform the steps performed by the device of one or more of Aspects 27-30.

Aspect 47: A device, comprising a memory and one or more processors coupled to the memory, the one or more processors configured to perform the steps performed by the device of one or more of Aspects 27-30.

Aspect 48: An apparatus, comprising at least one means for performing the steps performed by the device of one or more of Aspects 27-30.

Aspect 49: A non-transitory computer-readable medium storing code, the code comprising instructions executable by a processor to perform the steps performed by the device of one or more of Aspects 27-30.

Aspect 50: A method comprising the steps performed by the device of one or more of Aspects 27-30.

The foregoing disclosure provides illustration and description but is not intended to be exhaustive or to limit the aspects to the precise forms disclosed. Modifications and variations may be made in light of the above disclosure or may be acquired from practice of the aspects.

As used herein, the term “component” is intended to be broadly construed as hardware and/or a combination of hardware and software. “Software” shall be construed broadly to mean instructions, instruction sets, code, code segments, program code, programs, subprograms, software modules, applications, software applications, software packages, routines, subroutines, objects, executables, threads of execution, procedures, and/or functions, among other examples, whether referred to as software, firmware, middleware, microcode, hardware description language, or otherwise. As used herein, a “processor” is implemented in hardware and/or a combination of hardware and software. It will be apparent that systems and/or methods described herein may be implemented in different forms of hardware and/or a combination of hardware and software. The actual specialized control hardware or software code used to implement these systems and/or methods is not limiting of the aspects. Thus, the operation and behavior of the systems and/or methods are described herein without reference to specific software code, since those skilled in the art will understand that software and hardware can be designed to implement the systems and/or methods based, at least in part, on the description herein.

As used herein, “satisfying a threshold” may, depending on the context, refer to a value being greater than the threshold, greater than or equal to the threshold, less than the threshold, less than or equal to the threshold, equal to the threshold, not equal to the threshold, or the like.

Even though particular combinations of features are recited in the claims and/or disclosed in the specification, these combinations are not intended to limit the disclosure of various aspects. Many of these features may be combined in ways not specifically recited in the claims and/or disclosed in the specification. The disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a, b, c, a+b, a+c, b+c, and a+b+c, as well as any combination with multiples of the same element (e.g., a+a, a+a+a, a+a+b, a+a+c, a+b+b, a+c+c, b+b, b+b+b, b+b+c, c+c, and c+c+c, or any other ordering of a, b, and c).

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the terms “set” and “group” are intended to include one or more items and may be used interchangeably with “one or more.” Where only one item is intended, the phrase “only one” or similar language is used. Also, as used herein, the terms “has,” “have,” “having,” or the like are intended to be open-ended terms that do not limit an element that they modify (e.g., an element “having” A may also have B). Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A user device, comprising:
a pressure sensor configured to detect a pressure at an interface between a body of a user and the user device;
an output component; and
one or more processors configured to:
monitor, in connection with a biometric measurement operation and using the pressure sensor, whether a pressure at the interface between the body of the user and the user device is within a range used for obtaining data for a biometric measurement;
provide, via the output component, an indication of whether the user is to adjust the pressure based on whether the pressure is within the range; and
obtain the data for the biometric measurement.

2. The user device of claim 1, wherein a first side of the user device includes a display, and
wherein a second side of the user device, opposite the first side, includes the pressure sensor.

3. The user device of claim 1, wherein the user device is a wearable device configured for attachment to an arm of the user, and
wherein the user device further comprises a rotatable element configured to cause actuation of the pressure sensor to adjust the pressure at the interface between the body of the user and the user device.

4. The user device of claim 3, wherein the rotatable element is a knob, or a bezel that surrounds a display of the user device.

5. The user device of claim 1, wherein the one or more processors, to obtain the data for the biometric measurement, are configured to:
obtain the data for the biometric measurement using photoacoustic signal generation.

6. The user device of claim 1, wherein the one or more processors, to obtain the data for the biometric measurement, are configured to:
obtain first data for the biometric measurement while the pressure is outside of the range and second data for the biometric measurement while the pressure is within the range; and
determine the biometric measurement based on the second data regardless of the first data.

7. The user device of claim 1, wherein the one or more processors, to obtain the data for the biometric measurement, are configured to:
cause emission of an optical signal at the body of the user; and
adjust an intensity of the optical signal based on an amount by which the pressure is outside of the range.

8. The user device of claim 1, wherein the one or more processors are further configured to:
determine the biometric measurement based at least in part on the data.

9. The user device of claim 1, wherein the output component is a display, a speaker, or a vibration component.

10. The user device of claim 1, wherein the user device further comprises:
at least one strap to enable the user device to be worn on an arm of the user.

11. The user device of claim 1, wherein the biometric measurement is a blood pressure measurement, a heart rate measurement, a heart rate variability measurement, or a pulse oxygen measurement.

12. A method, comprising:
initiating, by a user device, a biometric measurement operation relating to a user of the user device;
monitoring, by the user device in connection with the biometric measurement operation and using a pressure sensor of the user device, whether a pressure at an interface between a body of the user and the user device is within a range used for obtaining data for a biometric measurement;
providing, by the user device via an output component of the user device, an indication of whether the user is to adjust the pressure based on whether the pressure is within the range; and
obtaining, by the user device, the data for the biometric measurement.

13. The method of claim 12, wherein the data is obtained using photoacoustic signal generation.

14. The method of claim 12, wherein the data that is obtained includes first data obtained while the pressure is outside of the range and second data obtained while the pressure is within the range, and
wherein the method further comprises:
determining the biometric measurement based on the second data regardless of the first data.

15. The method of claim 12, wherein obtaining the data comprises:
emitting an optical signal at the body of the user; and
adjusting an intensity of the optical signal based on an amount by which the pressure is outside of the range.

16. The method of claim 12, further comprising:
determining the biometric measurement based at least in part on the data.

17. The method of claim 12, wherein the output component is a display of the user device, a speaker of the user device, or a vibration component of the user device.

18. The method of claim 12, wherein the user device is a wearable device configured to be worn on an arm of the user.

19. The method of claim 12, wherein the biometric measurement is a blood pressure measurement, a heart rate measurement, a heart rate variability measurement, or a pulse oxygen measurement.

20. A user device, comprising:
means for initiating a biometric measurement operation relating to a user of the user device;
means for monitoring whether a pressure at an interface between a body of the user and the user device is within a range used for obtaining data for a biometric measurement;
means for providing an indication of whether the user is to adjust the pressure based on whether the pressure is within the range; and
means for obtaining the data for the biometric measurement.

21. The user device of claim 20, wherein the means for obtaining the data for the biometric measurement include:
means for obtaining the data for the biometric measurement using photoacoustic signal generation.

22. The user device of claim 20, wherein the means for obtaining the data for the biometric measurement include:
means for obtaining first data for the biometric measurement while the pressure is outside of the range and second data for the biometric measurement while the pressure is within the range; and
means for determining the biometric measurement based on the second data regardless of the first data.

23. The user device of claim 20, wherein the means for obtaining the data for the biometric measurement include:
means for emitting an optical signal at the body of the user; and
means for adjusting an intensity of the optical signal based on an amount by which the pressure is outside of the range.

24. The user device of claim 20, further comprising:
means for determining the biometric measurement based at least in part on the data.

25. The user device of claim 20, wherein the means for providing include a display of the user device, a speaker of the user device, or a vibration component of the user device.

26. The user device of claim 20, wherein the means for monitoring include a pressure sensor of the user device.

27. A non-transitory computer-readable medium storing a set of instructions, the set of instructions comprising:
one or more instructions that, when executed by one or more processors of a user device, cause the user device to:
initiate a biometric measurement operation relating to a user of the user device;
monitor, in connection with the biometric measurement operation and using a pressure sensor of the user device, whether a pressure at an interface between a body of the user and the user device is within a range used for obtaining data for a biometric measurement;
provide, via an output component of the user device, an indication that the user is to increase or decrease the pressure to within the range;
detect that the pressure is within the range used for obtaining the data for the biometric measurement; and
obtain the data for the biometric measurement based on detecting that the pressure is within the range.

28. The non-transitory computer-readable medium of claim 27, wherein the one or more instructions, that cause the user device to obtain the data for the biometric measurement, cause the user device to:
obtain the data for the biometric measurement using photoacoustic signal generation.

29. The non-transitory computer-readable medium of claim 27, wherein the one or more instructions further cause the user device to:
determine the biometric measurement based at least in part on the data.

30. The non-transitory computer-readable medium of claim 27, wherein the output component is a display, a speaker, or a vibration component.
